(19) United States
(12) Patent Application Publication
(10) Pub. No.: US 2020/0016333 A1
(43) Pub. Date: Jan. 16, 2020

(54) A MEDICAMENT DELIVERY DEVICE

(71) Applicant: Sanofi-Aventis Deutschland GMBH, Frankfurt am Main (DE)

(72) Inventors: Francisco Soares, Frankfurt am (DE); Bernhard Forys, Frankfurt am (DE); Stefan Verlaak, Paderno d`Adda (LC), (IT); Ilario Melzi, Milano (Mi) (IT)

(21) Appl. No.: 16/336,576

(22) PCT Filed: Sep. 20, 2017
(86) PCT No.: PCT/EP2017/073725

§ 371 Date: Mar. 26, 2019

(30) Foreign Application Priority Data
Sep. 27, 2016 (EP) 16190885.0

(51) Int. Cl. A61M 5/158; A61M 5/142

(52) U.S. Cl. CPC A61M 5/158; A61M 2005/1402; A61M 5/14248

(57) ABSTRACT

The present disclosure relates to a medicament delivery device. The medicament delivery device comprises a housing, a needle and a needle actuating mechanism. The needle actuating mechanism is configured to rotate the needle relative to the housing to move the needle from a stowed position to a primed position. The present invention also relates to a method of operating a medicament delivery device.

### CROSS REFERENCE TO RELATED APPLICATIONS

The present application is the national stage entry of International Patent Application No. PCT/EP2017/073725, filed on Sep. 20, 2017, and claims priority to Application No. EP 16190885.0, filed on Sep. 27, 2016, the disclosures of which are incorporated herein by reference.

### TECHNICAL FIELD

The present disclosure relates to a medicament delivery device and to a method of operating a medicament delivery device.

### BACKGROUND

A variety of diseases exist that require regular treatment by injection of a medicament and such injections can be performed by using injection devices. Various injection devices for delivering injections of medicament are known in the art. Another type of injection pump that is gaining traction is the bolus injector device. Some bolus injector devices are intended to be used with relatively large volumes of medicament, typically at least 1 ml and maybe a few ml. Injection of such large volumes of medicament can take some minutes or even hours. Such high capacity bolus injector devices can be called large volume devices (LVDs). Generally such devices are operated by the patients themselves, although they may also be operated by medical personnel.

### SUMMARY

In some aspects, an improved medicament delivery device and an improved method of operating a medicament delivery device are provided.

According to one aspect, there is provided a medicament delivery device comprising: a housing; a needle; and, a needle actuating mechanism configured to rotate the needle relative to the housing to move the needle from a stowed position to a primed position.

The medicament delivery device is easier to store when the needle is in the stowed position because the needle takes up less space in a particular direction of the medicament delivery device. The needle only needs to be moved to the primed position when medicament is to be delivered to the patient. Therefore, the dimension of the housing in said direction can be reduced when the medicament delivery device is not in use to save space.

In one embodiment, the needle actuating mechanism comprises a needle holder that is rotatable relative to the housing to move the needle from the stowed position to the primed position. The needle may be slidably mounted to the needle holder.

In one embodiment, the needle actuating mechanism comprises a needle holder that is rotatable relative to the housing to move the needle from the stowed position to the primed position, wherein the needle actuating mechanism is configured to slide the needle relative to the needle holder to move the needle in a linear path relative to the housing from the primed position to an extended position wherein the needle projects out of the housing.

In one embodiment, the needle actuating mechanism comprises first and second arms that are each pivotally coupled to the housing and pivotally coupled to the needle holder. The first and second arms may be pivotally coupled to the needle holder proximate to respective first and second ends of the needle holder.

In one embodiment, the first and second arms overlap in the direction of the longitudinal axis of the needle when the needle is in the stowed position.

In one embodiment, the first and second arms are substantially parallel to the longitudinal axis of the needle when the needle is in the stowed position. This helps to reduce the amount of space taken up by the first and second arms and the needle in a direction perpendicular to the longitudinal axis of the needle when the needle is in the stowed position.

In one embodiment, the first and second arms are configured to rotate in opposite directions to move the needle from the stowed position to the primed position.

The first and second arms may rotate in opposite directions when the needle moves from the primed position to the stowed position.

In one embodiment, the medicament delivery device further comprises a flexible conduit connected to the needle for fluidly communicating the needle with a medicament reservoir. The flexible conduit allows for the needle to remain fluidly connected to the medicament reservoir when the needle is moved between the stowed and primed positions.

The needle actuating mechanism may be configured to move the needle relative to the housing from the primed position to an extended position wherein the needle projects out of the housing. Therefore, the needle may be protected by the housing when in the primed position and/or may automatically enter an injection site of the patient when moved from the primed position to the extended position. The needle may move in a linear path from the primed position to the extended position.

In one embodiment, the housing comprises a distal wall and the needle extends substantially parallel to the distal wall when the needle is in the stowed position. This allows for the dimension of the housing in a direction perpendicular to the distal wall to be reduced when the needle is in the stowed position.

In one embodiment, the needle is rotated substantially 90 degrees relative to the housing from the stowed position to the primed position.

In one embodiment, the medicament delivery device is a large volume device.

The medicament delivery device may further comprise a medicament reservoir that contains a medicament.

According to another aspect, there is also provided a method of operating a medicament delivery device comprising a housing, a needle and a needle actuating mechanism, the method comprising: positioning a distal end of the housing in proximity to an injection site of a patient;

and, operating the needle actuating mechanism to rotate the needle relative to the housing to move the needle from a stowed position to a primed position.

In one embodiment, the needle actuating mechanism comprises a needle holder that is rotatable relative to the housing to move the needle from the stowed position to the primed position, wherein the needle actuating mechanism is configured to slide the needle relative to the needle holder to move the needle in a linear path relative to the housing from the primed position to an extended position wherein the needle projects out of the housing.

In one embodiment, the method comprises operating the needle actuating mechanism to slide the needle relative to the needle holder to move the needle in a linear path relative to the housing from the primed position to the extended position.

These and other aspects of the invention will be apparent from and elucidated with reference to the embodiments described hereinafter.

### BRIEF DESCRIPTION OF THE FIGURES

Embodiments of the invention will now be described, by way of example only, with reference to the accompanying drawings, in which:

FIG. 1 is a schematic cross-sectional side view of a medicament delivery device according to a first embodiment of the invention;

FIG. 2 is a schematic side view of part of the medicament delivery device of FIG. 1, wherein a needle is in a stowed position;

FIG. 3 is a schematic side view of part of the medicament delivery device of FIG. 1, wherein the needle is in a first intermediate position;

FIG. 4 is a schematic side view of part of the medicament delivery device of FIG. 1, wherein the needle is in a second intermediate position;

FIG. 5 is a schematic side view of part of the medicament delivery device of FIG. 1, wherein the needle is in a primed position;

FIG. 6 is a schematic side view of part of the medicament delivery device of FIG. 1, wherein the needle is in an extended position;

FIG. 7 is a schematic side view of part of a medicament delivery device according to a second embodiment of the invention, wherein a needle is in a stowed position;

FIG. 8 is a schematic side view of part of the medicament delivery device of FIG. 7, wherein the needle is in a primed position; and,

FIG. 9 is a schematic side view of part of the medicament delivery device of FIG. 1, wherein the needle is in an extended position.

### DETAILED DESCRIPTION

A medicament delivery device, as described herein, may be configured to inject a medicament into a patient. For example, delivery could be sub-cutaneous, intra-muscular, or intravenous.

Such a device could be operated by a patient or care-giver, such as a nurse or physician, and can include various types of safety syringe, pen-injector, or auto-injector. The device can include a cartridge-based system that requires piercing a sealed ampule before use. Volumes of medicament delivered with these various devices can range from about 0.5 ml to about 2 ml. Yet another device can include a large volume device (“LVD”) or patch pump, configured to adhere to a patient's skin for a period of time (e.g., about 5, 15, 30, 60, or 120 minutes) to deliver a “large” volume of medicament (typically about 2 ml to about 10 ml).

In combination with a specific medicament, the presently described devices may also be customized in order to operate within required specifications. For example, the device may be customized to inject a medicament within a certain time period (e.g., about 3 to about 20 seconds for auto-injectors, and about 10 minutes to about 60 minutes for a large volume device). Other specifications can include a low or minimal level of discomfort, or to certain conditions related to human factors, shelf-life, expiry, biocompatibility, environmental considerations, etc. Such variations can arise due to various factors, such as, for example, a drug ranging in viscosity from about 3 cP to about 50 cP. Consequently, a drug delivery device will often include a hollow needle ranging from about 26 to about 31 Gauge in size. Common sizes are 27 and 29 Gauge.

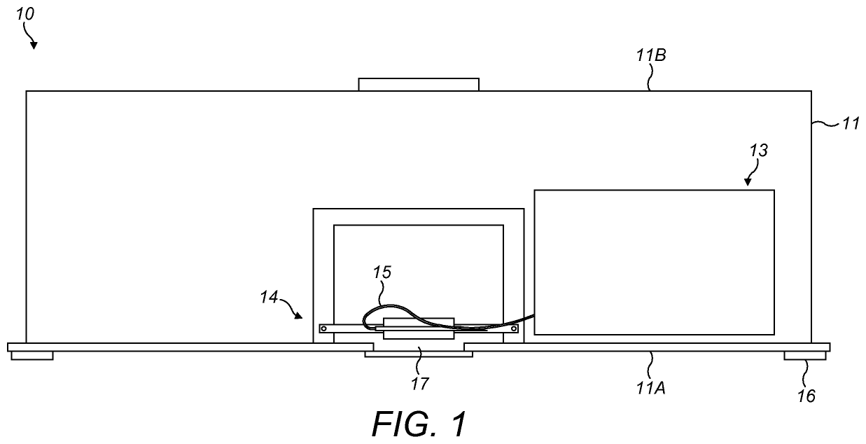

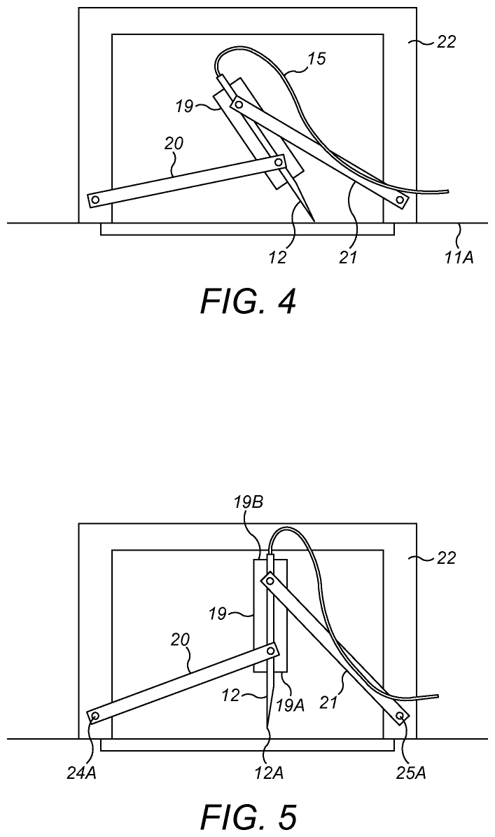

FIGS. 1 to 6 show a medicament delivery device 10, which in an exemplary embodiment comprises a bolus injector device, according to a first embodiment of the invention. The medicament delivery device 10 may be in the form of a large volume device. The medicament delivery device 10 comprises a housing 11, a needle 12 for injection of medicament into a patient's body, a medicament dispensing mechanism 13 and a needle actuating mechanism 14.

The medicament dispensing mechanism 13 and the needle actuating mechanism 14 are located inside the housing 11. The medicament dispensing mechanism 13 comprises a medicament reservoir (not shown) containing a supply of medicament to be administered to a patient. The medicament delivery device 10 further comprises a flexible conduit 15 that fluidly connects the medicament reservoir to the bore of the needle 12.

A number of the functional components of the medicament dispensing mechanism 13 are omitted for the sake of clarity and brevity. For example, although not shown in the figures, the medicament dispensing mechanism 13 may include one or more of the following components. A controller configured to control operation of the medicament delivery device 10. A medicament reservoir including, for example, a cartridge or a vial formed of glass. A plunger may be provided within the cartridge and plunger driver mechanically coupled to the plunger. The plunger driver may be controllable to move the plunger along the medicament cartridge. The force provided by the plunger causes medicament to be expelled through a medicament delivery aperture in the medicament cartridge and along the flexible conduit 15 to the needle 12 to be expelled through the bore of the needle 12. An electrical power source in the form of a battery to power to the controller. The battery may also electrically power the plunger driver, if this is an electrically driven device.

The housing 11 is generally cylindrical and comprises a distal wall 11A and a proximal wall 11B. The term “distal” refers to a location that is relatively closer to a site of injection and the term “proximal” refers to a location that is relatively further away from the injection site.

The outer surface of the distal wall 11A comprises an adhesive layer 16 that is initially covered by a label (not shown). In use, the label is removed from the adhesive layer 16 and then the adhesive layer 16 is stuck to the patient's skin at the injection site of the patient such that the distal wall 11A of the housing 11 is adhered to the injection site.

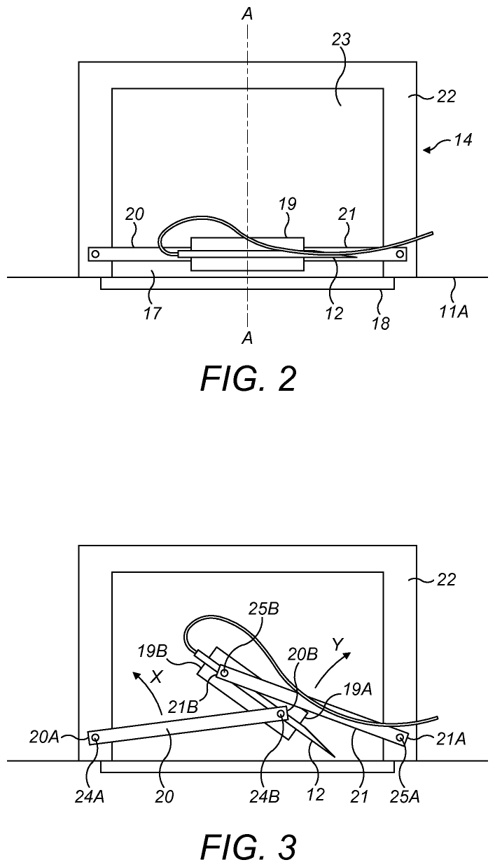

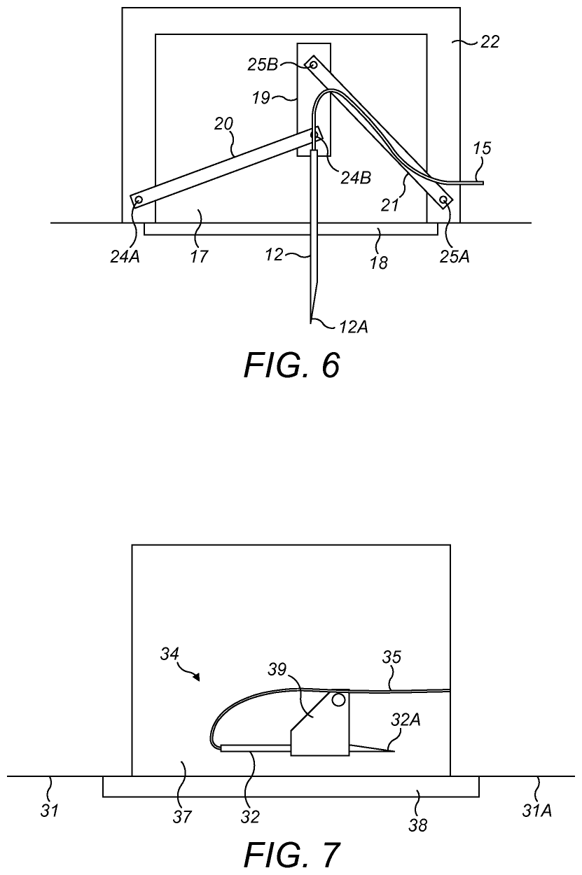

The distal wall 11A of the housing 11 includes an aperture 17 through which the needle 12 can project in use. The needle actuating mechanism 14 is configured to move the needle 12 from a stowed position (shown in FIGS. 1 and 2) to a primed position (shown in FIG. 5) and then to an extended position (shown in FIG. 6). In the stowed position, the needle 12 is disposed within the housing 11 of the medicament delivery device 10. In the extended position, the needle 12 projects from the distal wall 11A of the housing 11 through the aperture 17 so as to pierce and inject a patient's skin when the medicament delivery device 10 is attached to a patient.

The medicament delivery device 10 further comprises a septum 18 that is fixed to the distal wall 11A of the housing 11. The septum 18 is located over the aperture 17 in the distal wall 11A of the housing 11. The needle 12, which is initially in the stowed position, is protected by the septum 18. More specifically, the septum 18 prevents the ingress of contaminants through the aperture 17 in the distal wall 11A and into contact with the sterile needle 12. When the needle 12 is moved to the extended position, the needle 12 pierces the septum 18 and the end of the needle 12 passes through the septum 18 to project from the distal wall 11A. The septum 18 may be manufactured from an impermeable material such as plastic, rubber or metal foil.

The needle actuating mechanism 14 comprises a needle holder 19, first and second arms 20, 21, a drive mechanism (not shown) and a needle insertion mechanism (not shown). The needle holder 19 is configured to receive the needle 12 and is rotatable relative to the housing 11 to move the needle 12 from the stowed position to the primed position. The needle holder 19 is generally elongate and comprises a slot or aperture for slidably receiving the needle 12.

The housing 11 comprises an internal wall 22 that defines a chamber 23. The needle 12 and needle holder 19 are located in the chamber 23 when the needle 12 is in the stowed and primed positions.

A first end 20A of the first arm 20 is pivotally coupled to the internal wall 22 of the housing 11 by a first pivotal coupling 24A and a remote second end 20B of the first arm 20 is pivotally coupled to a distal end 19A of the needle holder 19 by a second pivotal coupling 24B. A first end 21A of the second arm 21 is pivotally coupled to the internal wall 22 of the housing 11 by a third pivotal coupling 25A and a remote second end 21B of the second arm 21 is pivotally coupled to a proximal end 19B of the needle holder 19 by a fourth pivotal coupling 25B. The first end 20A of the first arm 20 is pivotally coupled to the internal wall 22 on the opposite side of the central axis (shown by chain-dashed line ‘A-A’ in FIG. 2) of the housing 11 to the first end 21A of the second arm 21.

The needle 12 is initially in the stowed position, wherein the needle 12, needle holder 19 and the first and second arms 20, 21 are substantially parallel to the distal wall 11A of the housing 11 and substantially perpendicular to the central axis A-A of the housing 11. Moreover, the distal end 19A of the needle holder 19 is located nearest to the first end 21A of the second arm 21 and the proximal end 19B of the needle holder 19 is located nearest to the first end 20A of the first arm 20 such that the first and second arms 20, 21 overlap in the direction of the longitudinal axis of the needle 12, which is substantially perpendicular to the central axis A-A of the housing 11. In addition, the first, second, third and fourth pivotal couplings 24A, 24B, 25A, 25B are substantially aligned in a single plane that is substantially parallel to the distal wall 11A of the housing 11.

The first end 20A of the first arm 20 is coupled to the drive mechanism (not shown). The drive mechanism is configured to urge the first arm 20 to rotate relative to the internal wall 22 of the housing 11 in a first rotational direction (shown by arrow ‘X’ shown in FIG. 3) such that the first arm 20 swings through an arc relative to the housing 11 and thus the second end 20B of the first arm 20, and therefore the distal end 19A of the needle holder 19 coupled thereto by the second pivotal coupling 24B, rotates about the first pivotal coupling 24A and moves away from the distal wall 11A and towards the central axis A-A of the housing 11. The drive mechanism is operable by an actuator 26 which may be, for example, a button or switch that is located on the housing 11 and is connected to the drive mechanism. In one embodiment, the drive mechanism comprises an electric motor that is operated upon actuation of the actuator 26 to rotate the first arm 20 in the first rotational direction X. In an alternative embodiment, the drive mechanism comprises a locking mechanism and a biasing member, for example, a spiral or torsional spring, which is configured to bias the first arm 20 to rotate in the first rotational direction X. The locking mechanism initially retains the first arm 20 in position against the force of the biasing member. The locking mechanism is unlocked upon actuation of the actuator 26 such that the biasing member is released to rotate the first arm 20 in the first rotational direction X.

To move the needle 12 from the stowed position to the primed position, the patient actuates the actuator 26 to operate the drive mechanism. This causes the first arm 20 to rotate in the manner described above such that the second pivotal coupling 24B, and thus the distal end 19A of the needle holder 19 coupled thereto, rotates about the first pivotal coupling 24A to move away from the distal wall 11A and towards the central axis A-A of the housing 11. The proximal end 19B of the needle holder 19 is coupled to the internal wall 22 of the housing 11 by the second arm 21 such that, when the first arm 20 rotates in the first rotational direction X, the second arm 21 swings through an arc relative to the internal wall 22 such that the second arm 21 rotates in a second rotational direction (shown by arrow ‘Y’ in FIG. 3) opposite to the first rotational direction X. This causes the second end 21B of the second arm 21, and thus the proximal end 19B of the needle holder 19 coupled thereto by the fourth pivotal coupling 25B, to rotate about the third pivotal coupling 25A and move away from the distal wall 11A and towards the central axis A-A of the housing 11. Therefore, the distal and proximal ends 19A, 19B of the needle holder 19 rotate about the first and third pivotal couplings 24A, 25A respectively such that the needle holder 19 rotates relative to the housing 11 to move the needle 12 from the stowed position to the primed position.

The distance between the first and second pivotal couplings 24A, 24B and the distance between the third and fourth pivotal couplings 25A, 25B is kept constant by the first and second arms 20, 21. When the needle 12 is in the primed position, the second and fourth pivotal couplings 24B, 25B, and thus the distal and proximal ends 19A, 19B of the needle holder 19 that are attached thereto, to align in a direction perpendicular to the central axis A-A of the housing 11.

The needle actuating mechanism 14 is configured such that when the needle 12 is moved from the stowed position to the primed position the needle 12 is rotated such that the injection end 12A is pointed towards the injection site and thus the angle between the longitudinal axis of the needle 12 and the distal wall 11A of the housing 11 is increased. Therefore, when the needle 12 is in the initial stowed position the amount of space taken up by the needle 12 in the direction of the central axis A-A of the housing 11 is reduced, in comparison to a device wherein the angle of the needle 12 is fixed relative to the housing 11, and the needle 12 only needs to be moved to the primed position when an injection is to be performed. Therefore, the distance between the distal and proximal walls 11A, 11B of the housing 11 can be reduced when the medicament delivery device 10 is not in use to save space. For example, in one exemplary embodiment (not shown), the housing 11 is manufactured from separate distal and proximal parts that are moveably coupled together, for example, by a latch or screw thread, such that the housing 11 is collapsible when the medicament delivery device 10 is not in use to save space. When injection is required, the distal and proximal parts are moved away from each other such that the distance between the distal and proximal walls of the distal and proximal parts is increased to provide sufficient space to allow for the needle 12 to rotate. The needle actuating mechanism is then operated to move the needle from the stowed position to the primed position.

Movement of the needle 12 from the stowed position to the primed position is a complex motion, wherein the needle 12 and needle holder 19 are rotated relative to the housing 11 and are also translated such that the needle 12 and needle holder 19 are moved away from the distal wall 11A of the housing 11. In the exemplary embodiment shown in FIGS. 1 to 6, the needle 12 is rotated approximately 90 degrees relative to the housing 11 from the stowed position to the primed position such that when the needle 12 is in the stowed position the longitudinal axis of the needle 12 is substantially parallel to the distal wall 11A of the housing 11 and when the needle 12 is in the primed position the longitudinal axis of the needle 12 is substantially perpendicular to the distal wall 11A. However, it should be recognised that in alternative embodiments (not shown) the needle 12 and/or the needle holder 19 may have a different orientation relative to the housing 11 when the needle 12 is in the stowed position and/or the primed position.

The injection end 12A of the needle 12 and the distal end 19A of the needle holder 19 are located near to the aperture 17 in the distal wall 11A such that the injection end 12A faces towards the injection site of the patient when the needle 12 is in the primed position and the proximal end 19B of the needle holder 19 is remote from the distal wall 11A. Furthermore, the needle 12 and needle holder 19 are both fully received within the chamber 23 in the housing 11 and are sealed by the septum 18.

When the needle 12 reaches the primed position, the needle insertion mechanism (not shown) is operated to move the needle 12 from the primed position to the extended position. This causes the needle 12 to move with respect to the housing 11 and needle holder 19 such that the needle 12 moves through the aperture 17 in the distal wall 11A of the housing 11 to pass through the septum 18 and penetrate the injection site of the patient. The movement of the needle 12 with respect to the housing 11 and needle holder 19 may be linear.

In one embodiment (not shown), the needle insertion mechanism comprises an electric motor that is coupled to the needle 12 by a linear gear assembly, for example, a rack and pinion. Once the needle 12 reaches the primed position, the electric motor is operated to move the needle 12 linearly to the extended position. For example, the needle insertion mechanism may comprise a sensor, such as a light gate or displacement transducer, which detects when the needle 12 is in the primed position and sends a signal to operate the electric motor. The sensor may be coupled to a controller. Alternatively, the sensor may be omitted and instead the needle insertion mechanism operates the electric motor a predetermined time interval after the patient has actuated the actuator 26. In yet another embodiment (not shown), the medicament delivery device 10 comprises a second actuator that is actuated by the patient to operate the electric motor to move the needle 12 from the primed position to the extended position. The electric motor of the needle insertion mechanism may be the same motor as, or a different motor to, the electric motor of the needle drive mechanism.

Although in the above described embodiment the needle insertion mechanism comprises an electric motor, in an alternative embodiment (not shown) the needle insertion mechanism instead comprises a locking mechanism and a biasing member, for example, a spring or a portion of resilient material, which is configured to bias the needle 12 relative to the needle holder 19 into the extended position. The locking mechanism initially retains the needle 12 retracted into the needle holder 19. The locking mechanism is unlocked when the needle 12 is moved to the primed position such that the biasing member is released to move the needle 12 to the extended position.

The needle 12 is coupled to the reservoir (not shown) of the medicament dispensing mechanism 13 by the flexible conduit 15, which maintains fluid connection between the needle 12 and the reservoir during movement of the needle 12 from the stowed position to the extended position. When the needle 12 is in the extended position, the medicament delivery mechanism 13 is operated to deliver medicament to the needle 12, via the flexible conduit 15, such that the medicament is supplied to the injection site of the patient.

In one exemplary embodiment, the medicament delivery mechanism 13 comprises a pump that is operated when the needle 12 is moved to the extended position to supply medicament to the injection site. For example, the medicament delivery mechanism 13 may comprise a sensor, such as a light gate or displacement transducer, which detects when the needle 12 is in the extended position and sends a signal to operate the pump. The sensor may be coupled to a controller. Alternatively, the sensor may be omitted and instead the pump is operated a predetermined time interval after the patient has actuated the actuator 26. In yet another embodiment, the pump is operated upon further input by the patient, for example, by the patient pressing the actuator 26 a second time.

An exemplary operation of the medicament delivery device 10 will now be described. The medicament delivery device 10 is typically stored in a sterile packaging (not shown). The patient first removes the medicament delivery device 10 from the sterile packaging. When the medicament delivery device 10 is removed from the sterile packaging the needle 12 is in the stowed position (as shown in FIGS. 1 and 2).

The label (not shown) is then removed from the adhesive layer 16 on the distal wall 11A of the housing 11. The adhesive layer 16 is then adhered to the patient's skin at the injection site such that the distal wall 11A of the housing 11 is secured to the injection site.

The patient then presses the actuator 26 to operate the drive mechanism, which causes the needle 12 and needle holder 19 to rotate relative to the housing 11 (as shown in FIGS. 2 to 4) until the needle 12 is moved to the primed position (as shown in FIG. 5). When the needle 12 reaches the primed position, the needle insertion mechanism (not shown) is operated such that the needle 12 is slid relative to the needle holder 19 to penetrate the septum 18 such that the needle moves to the extended position (as shown in FIG. 6), wherein the needle 12 enters the injection site of the patient. The medicament dispensing mechanism 13 is then operated to supply medicament to the needle 12 to deliver medicament to the injection site of the patient.

Once delivery of medicament to the injection site of the patient is finished, for example, due to the reservoir being depleted of medicament or due to a predetermined time period elapsing since the beginning of the medicament delivery process, the needle insertion mechanism is operated to retract the needle 12 back into the housing 11. This causes the needle 12 to move back to the primed position. For example, the electric motor of the needle insertion mechanism may be operated in reverse to retract the needle 12 into the housing 11. Alternatively, a second locking mechanism unlocked to release a second biasing member of the needle insertion mechanism that urges the needle 12 relative to the needle holder 19 to retract the needle 12 into the housing 11. The medicament delivery device 10 may then be removed from the injection site of the patient.

In the above described embodiment the drive mechanism is coupled to the first arm 20 to drive the first arm 20 to rotate relative to the housing 11 in the first rotational direction X. However, in an alternative embodiment (not shown), the drive mechanism is instead coupled to the second arm 21 and is configured to drive the second arm 21 to rotate relative to the housing 11 in the second rotational direction Y. In yet another embodiment (not shown), the drive mechanism is coupled to both of the first and second arms 20, 21 and is configured to drive the first arm 20 to rotate in the first rotational direction X and the second arm 21 to rotate in the second rotational direction Y. For example, the drive mechanism may comprise a first electric motor coupled to the first arm 20 and a second electric motor coupled to the second arm 21.

Referring now to FIGS. 7 to 9, a medicament delivery device 30 according to a second embodiment of the invention is shown. The medicament delivery device 30 of the second embodiment is similar to the medicament delivery device 10 of the first embodiment, having a housing 31, a needle 32 and a medicament dispensing mechanism (not shown). A difference is that the needle actuating mechanism 14 of the first embodiment is omitted and is replaced with an alternative needle actuating mechanism 34.

The medicament dispensing mechanism and the needle actuating mechanism 34 are located inside the housing 31. The medicament dispensing mechanism comprises a medicament reservoir (not shown) containing a supply of medicament to be administered to a patient. The medicament delivery device 30 further comprises a flexible conduit 35 that fluidly connects the medicament reservoir to the bore of the needle 32.

The housing 31 is generally cylindrical and comprises a distal wall 31A and a proximal wall (not shown). The term “distal” refers to a location that is relatively closer to a site of injection and the term “proximal” refers to a location that is relatively further away from the injection site. The outer surface of the distal wall 31A comprises an adhesive layer (not shown) for adhering the distal wall 31A to the injection site of the patient. The adhesive layer is initially covered by a removable label (not shown).

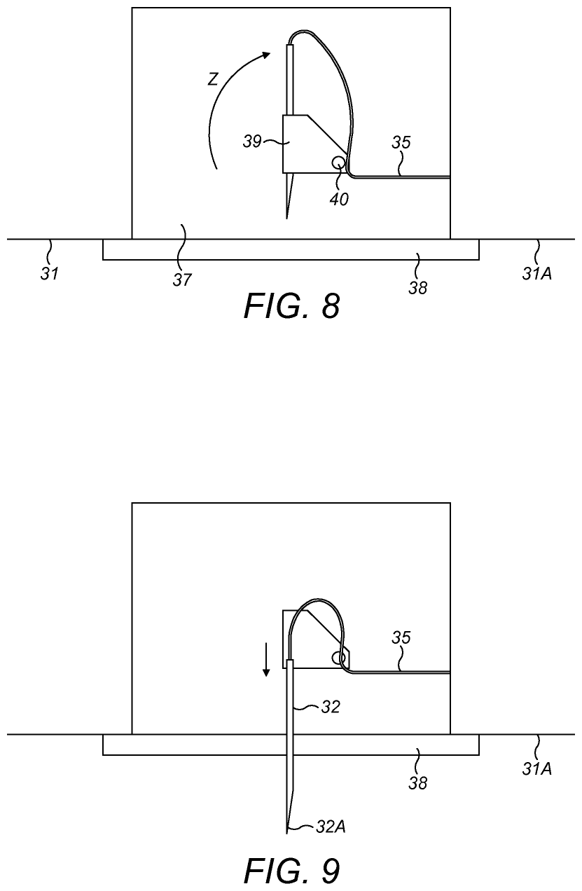

The distal wall 31A of the housing 31 includes an aperture 37 through which the needle 32 can project in use. A septum 38 is located over the aperture 37. The needle actuating mechanism 34 is configured to move the needle 32 from a stowed position (shown in FIG. 7) to a primed position (shown in FIG. 8) and then to an extended position (shown in FIG. 9). In the stowed position, the needle 32 is disposed within the housing 31 of the medicament delivery device 30. In the extended position, the needle 32 projects from the distal wall 31A of the housing 31 and through the aperture 37 so as to pierce the septum 38 and enter the patient's skin when the medicament delivery device 30 is attached to the patient.

The needle actuating mechanism 34 comprises a needle holder 39, a drive mechanism (not shown) and a needle insertion mechanism (not shown). The needle holder 39 is configured to receive the needle 32 and is rotatable relative to the housing 31 to move the needle 32 from the stowed position to the primed position. The needle holder 39 comprises a slot or aperture for slidably receiving the needle 32.

The needle holder 39 is connected to the housing 11 by a pivotal coupling 40 and is connected to the drive mechanism (not shown). The medicament delivery device 30 further comprises an actuator (not shown) that may be actuated by the patient to operate the drive mechanism.

The drive mechanism is configured to urge the needle holder 39 to rotate relative to the housing 11 in a first rotational direction (shown by arrow ‘Z’ in FIG. 8) from the initial stowed position to the primed position. Similarly to the first embodiment of the medicament delivery device 10, the drive mechanism of the medicament delivery device 30 of the second embodiment may comprise, for example, an electric motor (not shown) or a locking mechanism (not shown) that may be unlocked to release a biasing member that exerts a biasing force on the needle holder 39.

The needle actuating mechanism 34 is configured such that when the needle 32 is moved from the stowed position to the primed position the needle 32 is rotated such that the injection end 32A is pointed towards the injection site and thus the angle between the longitudinal axis of the needle 32 and the distal wall 31A of the housing 31 is increased. Therefore, when the needle 32 is in the stowed position the amount of space taken up by the needle 32 in the direction of the central axis of the housing 31 is reduced, in comparison to a device wherein the angle of the needle is fixed relative to the housing, and the needle 32 only needs to be moved to the primed position when an injection is to be performed. Therefore, the height of the housing 31, namely the distance between the distal wall 31A and the proximal wall of the housing 31, can be reduced when the medicament delivery device 30 is not in use to save space.

In the exemplary embodiment shown in FIGS. 7 to 9, the needle 32 is rotated approximately 90 degrees relative to the housing 31 from the stowed position to the primed position such that when the needle 32 is in the stowed position the longitudinal axis of the needle 32 is substantially parallel to the distal wall 31A of the housing 31 and when the needle 32 is in the primed position the longitudinal axis of the needle 32 is substantially perpendicular to the distal wall 31A. However, it should be recognised that in alternative embodiments (not shown) the needle 32 and/or needle holder 39 may have a different orientation relative to the housing 31 when the needle 32 is in the stowed position and/or the primed position.

Similarly to the first embodiment of the medicament delivery device 10, when the needle 32 of the medicament delivery device 30 of the second embodiment reaches the primed position the needle insertion mechanism (not shown) is operated to move the needle 32 from the primed position to the extended position. This causes the needle 32 to move with respect to the housing 31 and needle holder 39 such that the needle 32 moves through the aperture 37 in the distal wall 31A of the housing 31 to pass through the septum 38 and penetrate the injection site of the patient. The movement of the needle 32 with respect to the housing 31 and needle holder 39 may be linear.

The needle 32 is coupled to the reservoir (not shown) of the medicament dispensing mechanism (not shown) by the flexible conduit 35, which maintains fluid connection between the needle 32 and the reservoir during movement of the needle 32 from the stowed position to the extended position. When the needle 32 is in the extended position, the medicament delivery mechanism is operated to deliver medicament to the needle 32, via the flexible conduit 35, such that the medicament is supplied to the injection site of the patient.

An exemplary operation of the medicament delivery device 30 will now be described. The medicament delivery device 30 is typically stored in a sterile packaging (not shown). The patient first removes the medicament delivery device 30 from the sterile packaging. When the medicament delivery device 30 is removed from the sterile packaging the needle 32 is in the stowed position (as shown in FIG. 7). The label (not shown) is then removed from the adhesive layer (not shown) and the adhesive layer is adhered to the patient's skin at the injection site such that the distal wall 31A of the housing 31 is secured to the injection site.

The patient then actuates the actuator (not shown) to operate the drive mechanism, which causes the needle 32 and needle holder 39 to rotate relative to the housing 31 until the needle 32 is moved to the primed position (as shown in FIG. 8). When the needle 32 reaches the primed position, the needle insertion mechanism (not shown) is operated such that the needle 32 is slid relative to the needle holder 39 to penetrate the septum 38 such that the needle 32 moves to the extended position (as shown in FIG. 9), wherein the needle 32 enters the injection site of the patient. The medicament dispensing mechanism is then operated to supply medicament to the needle 32 to deliver medicament to the injection site of the patient.

Once delivery of medicament to the injection site of the patient is finished, for example, due to the reservoir being depleted of medicament or due to a predetermined time period elapsing since the beginning of the medicament delivery process, the needle insertion mechanism is operated to retract the needle 32 back into the housing 31 in a similar manner to the first embodiment of the medicament delivery device 10. This causes the needle 32 to move back to the primed position. The medicament delivery device 30 may then be removed from the injection site of the patient.

In the above described embodiments, the needle 12, 32 is retained fully within the housing 11, 31 when the needle 12, 32 is moved from the stowed position to the primed position such that the needle 12, 32 does not penetrate the septum 18, 38. However, in an alternative embodiment (not shown), the needle 12, 32 projects out of the housing 11, 31 when the needle 12, 32 is moved from the stowed position to the primed position and may penetrate the septum 18, 38. In one embodiment, the actuating mechanism 14, 34 is configured such that when the needle 12, 32 is moved to the primed position the needle 12, 32 projects out of the housing 11, 31 to the extent that the needle 12, 32 enters the injection site of the patient. In such an embodiment, it is not necessary for the needle 12, 32 to be moved to an extended position to delivery medicament to the injection site. In one such embodiment, the needle holder 19, 39 is omitted and instead the needle 12, 32 is rotatably coupled directly to the housing 11, 31.

In the above described embodiment, the medicament delivery device 10, 30 comprises a flexible conduit 15, 35 that fluidly communicates the needle 12, 32 with the medicament dispensing mechanism 13. However, in an alternative embodiment (not shown), the flexible conduit is omitted and instead a passage or rigid conduit is fluidly connected to the medicament dispensing mechanism. The needle comprises a channel that extends from the central bore of the needle. The channel is distal to the injection end of the needle and aligns with the rigid conduit when the needle is moved to the extended position to fluidly communicate the medicament dispensing mechanism with the bore of the needle.

The terms “drug” or “medicament” are used herein to describe one or more pharmaceutically active compounds. As described below, a drug or medicament can include at least one small or large molecule, or combinations thereof, in various types of formulations, for the treatment of one or more diseases. Exemplary pharmaceutically active compounds may include small molecules; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more of these drugs are also contemplated.

The term “drug delivery device” shall encompass any type of device or system configured to dispense a drug into a human or animal body. Without limitation, a drug delivery device may be an injection device (e.g., syringe, pen injector, auto injector, large-volume device, pump, perfusion system, or other device configured for intraocular, subcutaneous, intramuscular, or intravascular delivery), skin patch (e.g., osmotic, chemical, micro-needle), inhaler (e.g., nasal or pulmonary), implantable (e.g., coated stent, capsule), or feeding systems for the gastro-intestinal tract. The presently described drugs may be particularly useful with injection devices that include a needle, e.g., a small gauge needle.

The drug or medicament may be contained in a primary package or “drug container” adapted for use with a drug delivery device. The drug container may be, e.g., a cartridge, syringe, reservoir, or other vessel configured to provide a suitable chamber for storage (e.g., short- or long-term storage) of one or more pharmaceutically active compounds. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of a drug formulation (e.g., a drug and a diluent, or two different types of drugs) separately, one in each chamber. In such instances, the two chambers of the dual-chamber cartridge may be configured to allow mixing between the two or more components of the drug or medicament prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drug delivery devices and drugs described herein can be used for the treatment and/or prophylaxis of many different types of disorders. Exemplary disorders include, e.g., diabetes mellitus or complications associated with diabetes mellitus such as diabetic retinopathy, thromboembolism disorders such as deep vein or pulmonary thromboembolism. Further exemplary disorders are acute coronary syndrome (ACS), angina, myocardial infarction, cancer, macular degeneration, inflammation, hay fever, atherosclerosis and/or rheumatoid arthritis.

Exemplary drugs for the treatment and/or prophylaxis of diabetes mellitus or complications associated with diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the term “derivative” refers to any substance which is sufficiently structurally similar to the original substance so as to have substantially similar functionality or activity (e.g., therapeutic effectiveness).

Exemplary insulin analogues are Gly(A21), Arg(B31), Arg(B32) human insulin (insulin glargine); Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Exemplary insulin derivatives are, for example, B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin; B29-N-(N-lithocholyl-gamma-glutamyl)-des(B30) human insulin; B29-N-(ω-carboxyheptadecanoyl)-des(B30) human insulin and B29-N-(ω-carboxyhepta-decanoyl) human insulin. Exemplary GLP-1, GLP-1 analogues and GLP-1 receptor agonists are, for example: Lixisenatide/AVE0010/ZP10/Lyxumia, Exenatide/Exendin-4/Byetta/Bydureon/ITCA 650/AC-2993 (a 39 amino acid peptide which is produced by the salivary glands of the Gila monster), Liraglutide/Victoza, Semaglutide, Taspoglutide, Syncria/Albiglutide, Dulaglutide, rExendin-4, CJC-1134-PC, PB-1023, TTP-054, Langlenatide/HM-11260C, CM-3, GLP-1 Eligen, ORMD-0901, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, TT-401, BHM-034. MOD-6030, CAM-2036, DA-15864, ARI-2651, ARI-2255, Exenatide-XTEN and Glucagon-Xten.

An exemplary oligonucleotide is, for example: mipomersen/Kynamro, a cholesterol-reducing antisense therapeutic for the treatment of familial hypercholesterolemia.

Exemplary DPP4 inhibitors are Vildagliptin, Sitagliptin, Denagliptin, Saxagliptin, Berberine.

Exemplary hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Exemplary polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20/Synvisc, a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region.

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, and multispecific antibodies (e.g., diabodies, triabodies, tetrabodies), minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Exemplary antibodies are anti PCSK-9 mAb (e.g., Alirocumab), anti IL-6 mAb (e.g., Sarilumab), and anti IL-4 mAb (e.g., Dupilumab).

The compounds described herein may be used in pharmaceutical formulations comprising (a) the compound(s) or pharmaceutically acceptable salts thereof, and (b) a pharmaceutically acceptable carrier. The compounds may also be used in pharmaceutical formulations that include one or more other active pharmaceutical ingredients or in pharmaceutical formulations in which the present compound or a pharmaceutically acceptable salt thereof is the only active ingredient. Accordingly, the pharmaceutical formulations of the present disclosure encompass any formulation made by admixing a compound described herein and a pharmaceutically acceptable carrier.

Pharmaceutically acceptable salts of any drug described herein are also contemplated for use in drug delivery devices. Pharmaceutically acceptable salts are for example acid addition salts and basic salts. Acid addition salts are e.g. HCl or HBr salts. Basic salts are e.g. salts having a cation selected from an alkali or alkaline earth metal, e.g. Na+, or K+, or Ca2+, or an ammonium ion N+(R1)(R2)(R3)(R4), wherein R1 to R4 independently of each other mean: hydrogen, an optionally substituted C1 C6-alkyl group, an optionally substituted C2-C6-alkenyl group, an optionally substituted C6-C10-aryl group, or an optionally substituted C6-C10-heteroaryl group. Further examples of pharmaceutically acceptable salts are known to those of skill in the arts.

Pharmaceutically acceptable solvates are for example hydrates or alkanolates such as methanolates or ethanolates.

Those of skill in the art will understand that modifications (additions and/or removals) of various components of the substances, formulations, apparatuses, methods, systems and embodiments described herein may be made without departing from the full scope and spirit of the present invention, which encompass such modifications and any and all equivalents thereof.

1-17. (canceled)

18. A medicament delivery device comprising:
a housing;
a needle; and
a needle actuating mechanism configured to rotate the needle relative to the housing to move the needle from a stowed position to a primed position.

19. The medicament delivery device according to claim 18, wherein the needle actuating mechanism comprises a needle holder that is rotatable relative to the housing to move the needle from the stowed position to the primed position.

20. The medicament delivery device according to claim 19, wherein the needle is slidably mounted to the needle holder.

21. The medicament delivery device according to claim 19, wherein the needle actuating mechanism comprises first and second arms that are each pivotally coupled to the housing and pivotally coupled to the needle holder.

22. The medicament delivery device according to claim 21, wherein the first and second arms are pivotally coupled to the needle holder proximate to respective first and second ends of the needle holder.

23. The medicament delivery device according to claim 21, wherein the first and second arms overlap in a direction of a longitudinal axis of the needle when the needle is in the stowed position.

24. The medicament delivery device according to claim 21, wherein the first and second arms are substantially parallel to a longitudinal axis of the needle when the needle is in the stowed position.

25. The medicament delivery device according to claim 21, wherein the first and second arms are configured to rotate in opposite directions to move the needle from the stowed position to the primed position.

26. The medicament delivery device according to claim 18, further comprising a flexible conduit connected to the needle for fluidly communicating the needle with a medicament reservoir.

27. The medicament delivery device according to claim 18, wherein the needle actuating mechanism is configured to move the needle relative to the housing from the primed position to an extended position in which the needle projects out of the housing.

28. The medicament delivery device according to claim 27, wherein the needle moves in a linear path from the primed position to the extended position.

29. The medicament delivery device according to claim 18, wherein the housing comprises a distal wall, and the needle extends substantially parallel to the distal wall when the needle is in the stowed position.

30. The medicament delivery device according to claim 18, wherein the needle is rotated substantially 90 degrees relative to the housing from the stowed position to the primed position.

31. The medicament delivery device according to claim 18, wherein the medicament delivery device is a large volume device.

32. The medicament delivery device according to claim 31, wherein the large volume device is configured to contain at least 1 milliliter of medicament.

33. The medicament delivery device according to claim 18, further comprising a medicament reservoir that contains a medicament.

34. A needle actuating mechanism for a medicament delivery device, the needle actuating mechanism comprising:
a needle holder configured to receive a needle for the medicament delivery device, the needle holder being rotatable relative to a distal end of the medicament delivery device to move the needle from a stowed position to a primed position; and
an arm pivotably coupled to an end of the needle holder, the arm configured to rotate the end of the needle holder relative to the distal end of the medicament delivery device.

35. The needle actuating mechanism according to claim 34, wherein the arm is a first arm, the end is a first end, and the needle actuating mechanism comprises a second arm pivotably coupled to a second end of the needle holder.

36. A method of operating a medicament delivery device, the method comprising:
positioning a distal end of a housing of the medicament delivery device in proximity to an injection site of a patient; and,
operating a needle actuating mechanism of the medicament delivery device to rotate a needle of the medicament delivery device relative to the housing to move the needle from a stowed position to a primed position.

37. The method according to claim 36, wherein the needle actuating mechanism comprises a needle holder that is rotatable relative to the housing to move the needle from the stowed position to the primed position, wherein the needle actuating mechanism is configured to slide the needle relative to the needle holder to move the needle in a linear path relative to the housing from the primed position to an extended position wherein the needle projects out of the housing.
